(19) United States
(12) Patent Application Publication
(10) Pub. No.: US 2011/0009720 A1
(43) Pub. Date: Jan. 13, 2011

(54) Continuous whole blood glucose monitor

(76) Inventors: Kislaya Kunjan, Indianapolis, IN (US); Frank Perry Lloyd, Indianapolis, IN (US)

Correspondence Address: JAMES MINERVE, 115 Saddle Blanket Trial, Buda, TX, 78610, US

(21) Appl. No.: 11/982,565

(22) Filed: Nov. 2, 2007

Related U.S. Application Data
(60) Provisional application No. 60/856,456, filed on Nov. 2, 2006.

(51) Int. Cl. A61B 5/00; A61B 5/15; A61M 1/38; A61M 1/34

(52) U.S. Cl. 600/316; 600/310; 600/581; 604/6.04; 604/6.09

(57) ABSTRACT

A portable continuous whole blood glucose monitor comprising, a mid-infrared quantum cascade laser and driver in optical communication with a transmission cell and a photo-conductive detector and pre-amplifier. The monitor further comprises a peristaltic pump connected to a single lumen catheter peripherally inserted into a patient's vein. The single lumen catheter, in combination with the peristaltic pump, is operable to automatically withdraw a fixed and metered amount of whole blood from a patient, then a tube delivers a fixed and metered amount of the saline/surfactant supply to the whole blood. Methods of enhancing measurement sensitivity are also provided.

### CROSS REFERENCE TO RELATED APPLICATIONS

This application makes reference to Provisional Application 60/856,456, filed on Nov. 2, 2006.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### REFERENCE TO A SEQUENCE LISTING

Not Applicable

### BACKGROUND OF THE INVENTION

1. Field of the Invention

This invention relates generally to the measurement of biological parameters through spectroscopy; and more particularly, the invention relates to measurement of glucose using mid-infrared spectroscopy.

2. Description of Related Art

Recent medical studies have made it overwhelmingly clear that tight control of blood glucose levels of patients in critical care settings result in significant improvement in health outcomes. The adverse effect of hyperglycemia on hospital length of stay, morbidity, and mortality is substantial. Consequently, there is a nationwide pursuit of implementing improved glycemic control in both diabetic and non-diabetic hospitalized patients. At the core of tight glycemic control by intensive insulin therapy is frequent and accurate glucose monitoring. Part of the pursuit has focused on using infrared spectroscopy. Researchers realize that the mid-infrared region is very well suited for biological sensing due to its unique specificity for identifiable molecules of interest. Until recently, work in this region was limited due to lack of high powered light sources.

Infrared (IR) Spectroscopy has been the most active area in non-invasive and minimally invasive monitoring research. A lot of interest was generated by this technology many years ago when it was found that IR waves could be used to directly measure glucose. A brief background on the science of spectroscopy is now provided.

Spectroscopy is the use of the absorption, emission, or scattering of electromagnetic radiation by matter to qualitatively or quantitatively study matter or to study physical processes. Matter can be atoms, molecules, atomic or molecular ions, or solids and it can capture electromagnetic radiation and convert the energy of a photon to internal energy. Energy is transferred from the radiation field to the absorbing species. The energy change of the absorber can be described as a transition or an excitation from a lower energy level to a higher energy level.

Measuring the concentration of an absorbing species in a sample is accomplished by applying the Beer-Lambert law. The Beer-Lambert law defines a linear relationship between absorbance and concentration of an absorber of electromagnetic radiation. Assuming that the absorbance of a particular analyte is overlapped by absorbance from other constituents, the general form of the Beer-Lambert law is usually written as:

A=ΣεᵢCᵢL, Equation 1:

Where A is the absorbance, C is the molar absorptivity, C is the concentration of the constituent, L is the optical path length and the subscript ‘i’ corresponds to the constituent in the absorbing compound. The expression relating the concentration to IR absorption intensities then takes the following expanded form:

Cᵢ=K₀ᵢ+K₁ᵢA(λ)₁+K₂ᵢA(λ)₂+ . . . +K_{Ni}A(λ)N, Equation 2:

where K_{Ni} are the calibration coefficients for the ith constituent, and λ_{N} are the corresponding analytical wavelengths.

Experimental measurements are usually made in terms of transmittance (T).

A=−log(T)=−log(I/I₀), Equation 3:

where I and Iₒ represent light intensities before and after passing through the sample of path length ‘L’. The attenuation in intensity can be evaluated as a function of wavelength in order to extract information from the spectrum concerning the presence of the analyte in the sample (FIG. 1).

### Optical Techniques

Various optical techniques for glucose measurement, currently under development involve near infrared spectroscopy, mid-infrared spectroscopy, ram an spectroscopy, photo-acoustic spectroscopy, and scatter and polarization changes. Some of the daunting challenges posed by these techniques include weak optical signals, biochemical interference, and patient-to-patient variability. The mid-infrared and near infrared regions are relatively more useful in monitoring of analytes such as glucose.

The high initial cost of implementing mid-infrared technology can be overcome with widespread commercial applications. In particular, applications that place a premium on sensitivity, specificity and overall system accuracy, the spectral region of choice is the mid-infrared. Thus far, mid-infrared spectroscopy based systems have not been implemented in a clinical setting.

### Mid-Infrared Glucose Spectroscopy

Substances such as glucose have covalent bonds with fundamental resonance frequencies in the mid-infrared region of the light spectrum, i.e., at frequencies corresponding to infrared light wavelengths from 2.5 to 25 μm. Hence, the mid-infrared region of the absorption spectrum of such analytes contain relatively narrow absorption lines specific to each individual substance. Infrared spectroscopic technologies measure blood analyte levels (such as blood glucose levels) by measuring light absorption when an infrared spectrum is transmitted through a sample. Every chemical entity absorbs infrared light in a unique way, so every chemical entity has its own particular infrared spectrum. Moreover, the absorption of light is directly proportional to the concentration of the particular chemical entity in the test sample. Because each analyte (such as glucose) has its own unique infrared spectrum, it can be identified and measured. A glucose molecule belongs to the class of carbohydrates with atoms C, H and O in the ratio of 1:2:1. The strongest absorption bands involve stretching of the C—O bonds of COH and COC groups.

Glucose has fundamental absorption bands in the 9-10 μm region. Factors to consider when selecting which wavelength bands to use to measure glucose in biological fluid may include:
(i) Ensuring that the absorption band includes a strong absorption peak of glucose with minimal interference from substances such as urea and lactate, (the “glucose” band).
(ii) The wavelength band includes a region where glucose has negligible absorption (the “baseline” band).
(iii) Absorption bands for interferents where glucose has negligible absorption (“the interference” bands).

The measurement of blood glucose by any technique is inherently complex because of the wide range of potentially interfering components. For a noninvasive technique, not only are there many analytes within human blood that could interfere with the measurement (including the highly absorbing nature of water), but there are also other problems such as the variability, lack of homogeneity of human skin and the constantly changing human physiology. Present methods for monitoring blood glucose levels either require frequent physical sampling of the blood through finger pricks or manual blood removal from a vein using a syringe. Non-invasive optical methods under development do not require physical contact of the sample with the sensing element. However, these methods face serious challenges with regard to specificity and therefore measurement accuracy. Alternate measuring devices utilize subcutaneously implanted sensors to determine glucose levels in the interstitial fluid space. These measurements however suffer from an inherent time lag with the glucose levels in the blood, and associated inaccuracies. There continues to be an unmet need for an automated continuous “blood“glucose monitoring system that is accurate in the critical care settings.

Further, as disclosed in U.S. patent application Ser. No. 10/692,996 to Gore et al. (the “Gore Application”), the use of mid-infrared electromagnetic radiation may be used to sense glucose levels in ultrafiltered blood. Ultrafiltration is a variety of membrane filtration in which hydrostatic pressure forces a liquid against a semipermeable membrane, whereby suspended solids and solutes of high molecular weight (e.g. large protein molecules) are retained, while water and low molecular weight solutes (such as glucose) pass through the membrane. The latter can then be used for analysis. However, using the technique disclosed in the Gore Application has proven difficult in a clinical setting due to problems with their ultrafiltrate harvesting method and the low sensitivity of the system. The low sensitivity in part is due to their use of low powered thermal light sources and therefore having to contend with a low optical path length because that is the only way to perform any measurement in the water window of the mid-infrared spectrum. Additionally, reduction of noise from this method required use of cryogenically cooled detection apparatus that is bulky, and cumbersome. Further, as with many other glucose monitors utilizing optical methods, the method disclosed in the Gore Application works with only ultrafiltered blood products, as whole blood contains a number of cellular components that have made it very difficult to reproduce accurate readings. Further, U.S. Pat. No. 6,737,351 to Lendl et al. (the “Lendl Patent”) describes the use of mid-infrared quantum cascade laser for biological measurements, but does not disclose any method to carry out direct analytical measurements in whole blood samples.

Absorbance spectroscopy measurements can be carried out through two common modes, namely transmittance and reflectance or Attenuated Total Reflection (ATR). In the ATR mode light undergoes total internal reflection and an evanescent light wave penetrates into the sample. The absorption which thereby occurs leads to an attenuation of the intensity of the light transported. This attenuation in intensity can be evaluated as a function of wavelength in order to extract information from the spectrum concerning the presence of the analyte in the sample. For ATR measurement on whole blood, protein deposition on the surface of the crystal is a significant problem. Additionally, it has often been difficult to attain a high pathlength in an ATR mode. These shortcomings can be overcome in the transmission mode.

Blood contains about 45% cellular components including erythrocytes, leukocytes, and platelets; the remaining 55% are contributed by water and dissolved solids (3% of the total). Scientific studies have described the feasibility of whole blood based glucose measurement using the laboratory based Fourier Transform Infrared (“FTIR”) spectrometer. However, issues related to the bulky instrumentation, operational handling, fluidics and the optical scattering due to the blood cells have precluded the commercial adaptation of FTIR instruments for automated clinical analysis. In the past, high intensity lead salt lasers in the mid-infrared fingerprint region were bulky, required cryogenic cooling and were not explored for bio-sensing applications. In FTIR spectrometry, the incandescent light source behaves like a black body source and the optical path length through a sample must be in the low micrometer range (typically between 10-50 μm) in order to perform measurements in the finger print region. The short optical path length is a serious limitation on the sensitivity and therefore to achieve higher sensitivity, a higher intensity light source is highly desirable.

Given the state of the prior art the present invention has the following objectives:

measure venous blood glucose levels of patients in critical care settings in an automated fashion;

overcome the drawbacks associated with incandescent light sources;

discover the ideal mid-infrared path length which allows for resolvable differences between physiological concentrations of the analyte, such as glucose;

develop a sophisticated approach for estimating analyte concentrations in a complex mixture, such as whole blood using mid-infrared spectroscopy;

derive a technique to accurately find the optimal set of wavelengths in a spectral region, particularly for complex specimens where many wavelength terms may be required;

design a mid-infrared monitor that can be implemented in a clinical setting;

resolve the common problems experienced in the-prior art in using ATR and FTIR in the mid-infrared region in attempting to monitor glucose in a clinical setting;

invent a convenient and less intrusive technique for continuously extracting whole blood samples from a patient in a ICU setting;

devise a convenient method for calibrating the system yielding accurate results for individual patients; and

produce a monitor meeting the foregoing objective, is cost effective to implement in a clinical setting that operates at room temperature, does not require cryogenic cooling, is not bulky and cumbersome to operate, nor occupies a lot of space.

Therefore, in light of the foregoing, a blood glucose monitor that can continuously monitor the blood glucose levels of an ICU patient in an automated fashion, using venous whole blood as the sample medium would be greatly appreciated in the art. It may be noted here that the term continuous as used through out this application refers to a fluid sampling and glucose testing frequency that ranges from few seconds to several minutes between measurements. The use of a similar device utilized for sensing other blood analytes would be further appreciated.

### BRIEF SUMMARY OF THE INVENTION

The present invention provides a system and method for monitoring glucose levels in whole blood and other biological fluids like plasma or ultrafiltrate in patients, wherein blood glucose is monitored from whole blood samples taken automatically at predetermined intervals and tested utilizing mid-infrared spectroscopy. Non-ionic surfactants are utilized to homogenize samples through cell lysis, thereby allowing the use of unfiltered whole blood to be used, and providing for automated sensing using mid-infrared laser technology that can fit well within an intensive care unit.

Other objects and advantages of the present invention will be readily apparent upon a reading of the following brief descriptions of the drawing figures, detailed descriptions of preferred embodiments of the invention, the appended claims and drawings.

### BRIEF DESCRIPTION OF THE SEVERAL VIEWS OF THE DRAWINGS

The above mentioned and other objects and features of this invention and the manner of attaining them will become apparent, and the invention itself will be best understood by reference to the appended drawings. In the course of the following detailed description, reference will be made to the appended drawings in which:

FIG. 1; A Schematic of Light Energy Transmittance through a Sample

FIG. 2; A Schematic of the Entire Optical and Electronic Setup of an Embodiment of the Invention

FIG. 3; A Schematic of a the Complete System Interfaced with a Patient

FIG. 4; A Schematic of an Embodiment of the Invention Utilizing Fiber Optic Transmission Probes

FIG. 5; A Schematic of another Embodiment of the Invention Utilizing Fiber Optic Transmission Probes

FIG. 6; A Schematic of another Embodiment of the Invention Utilizing Fiber Optic ATR Probes;

FIG. 7; A plot of the real time signal response of the mid infrared QCL based prototype for spiked Whole Blood samples

FIG. 8; A plot of the real time signal response of the mid infrared QCL based prototype for spiked serum samples

FIG. 9; A graph of the Absorption Spectra of Glucose and Common Interferences taken using a FTIR Spectrometer

### DETAILED DESCRIPTION OF THE INVENTION

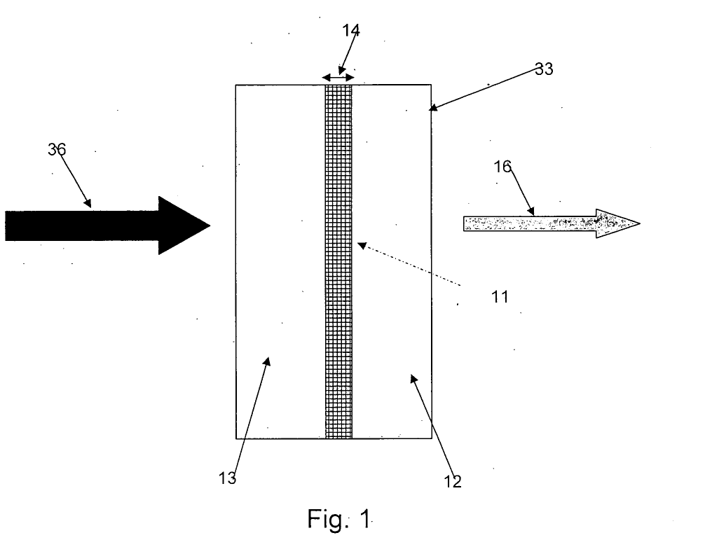

In the following description, like reference characters designate like or corresponding parts throughout the several views. Referring now to the drawings in detail, reference is made to FIGS. 1, 2 and 3. The present invention is a system and method for monitoring patients' glucose levels in whole blood and other biological fluids like plasma or ultrafiltrate. A preferred embodiment of the system comprises a mid-infrared, monochromatic, pulsed, multimode quantum cascade laser 21 (Laser Components, Germany), operating at around room temperature. The laser 21 comprises a driver (not shown) with a pulsed trigger 22. The laser 21 is capable of generating an intensity 15 having a wavelength of 9.65 μm, a pulse frequency of 10 kHz, a pulse width of 100 ns, and a peak power of 1000 mW. The system further comprises a room temperature Mercury Cadmium Telluride (MCT) photoconductive detector 30 with an integrated preamplifier (Vigo System, Poland) (not shown). The system comprises a thermo-electric cooler module 31 for maintaining the laser 21 and the detector 30 around room temperature. Both the laser 21 and detector 30 modules are shielded from the electromagnetic radiation intensities 15 and 16 by the thermo-electric cooler module 31.

The system comprises a fluidic system comprising a peristaltic pump 32, a demountable transmission based flow-cell (also transmission cell) 33, and a single lumen peripheral intravenous blood access catheter 40 for transmitting a whole blood sample 11 from a patient's peripheral vein (not shown) to the flow-cell 33, as in FIG. 1, and a tube 84 for carrying the surfactant and saline supply 50 to the mixer 41.

The system comprises a module 23 including a Gated Integrator (not shown), Boxcar Averager (not shown), and External Frequency Doubler for Active Baseline Subtraction (not shown). The signal from the integrated detector package 29 is fed to a Gated Integrator and Boxcar Averager System. The Gated Integrator/Boxcar Averager (hereafter referred to as the GI) is designed to recover fast, repetitive, analog signals. In the preferred embodiment, a time “gate” (not shown) of predetermined width is precisely positioned relative to the external trigger (provided from the laser driver) to coincide with the detector 30 sensor, which converts the electromagnetic radiation signal 16 to an electronic analog signal (not shown). The GI amplifies and integrates the analog signal that is present during the time the gate is open, ignoring noise and interference that are present at other times. The integrated signal 29 is then fed to a Boxcar Averager, which averages the output of the gated integrator over many shots from the laser 21.

Since any electromagnetic radiation signal, 16 present during time the gate is open, will add linearly, while noise will add in a “random walk” fashion as the square root of the number of shots, averaging N shots will improve the signal-to-noise ratio by a factor of the square root of N. In addition to using the averaging feature of the GI module, a unique Active Baseline Subtraction (ABS) module (not shown) is used which allows for actively canceling baseline drift. This overall method of signal processing is superior to the methods used by all prior researchers working on QCL based systems. Most use a lock-in amplifier module with or without an optical chopper. This is a sub-optimal solution for recovering fast analog signal from noisy backgrounds that is often typical of room-temperature QCLs. The output 26 from the GI module is read through a Data Acquisition Device (DAQ) device and processed using an algorithm 27. The algorithm continuously acquires and processes the data. After an initial calibration, the software displays the glucose read-out 28 on a real-time basis.

In its simplest form, the calibration problem for optical glucose measurement can be stated as: Given a set of optical measurements and corresponding glucose concentrations, develop a model which will allow prediction of glucose concentration based on analysis of future similar optical measurements. In the preferred embodiment, a single fixed QCL laser wavelength, specific to glucose has been implemented as a starting point. The univariate model is of the following form:

C_{Glucose}=K₀+K_{1.} A_{9.65 μm} Equation 1:

where Kᵢ are the calibration coefficients and A_{9.65μm} is the absorbance of the whole blood sample at 9.65 μm. The coefficients were determined by a two point calibration, i.e. by calculating absorbance of only the high end of the blood glucose concentration with reference to a blank. Linearity was assumed in the glucose concentration range of 0-500 mg/dl, by the use of the strongest analyte absorption band. Elaborate experimental studies using a variety of potential interferents have been performed by the applicant and the absorbance at 9.65 μm was highly specific to glucose.

Still referring to FIGS. 1, 2 and 3, in another preferred embodiment the system uses multiple wavelengths in the mid-infrared spectrum to accurately quantify physiologically changing blood glucose levels in hospitalized patients. Referring now to FIGS. 1, 2 and 3, in another preferred embodiment, the system uses a highly miniaturized tunable quantum cascade laser 21, such as a miniature QCL from Daylight Solutions, CA. The tunable laser is optionally centered at or near 9.4 μm having a ±5 percent tunability around the center wavelength (i.e. from 8.93 to 9.87 μm). The system comprises a miniaturized thermoelectrically cooled photoconductive MCT detector package 31 optimized for use with the tunable laser 21 system. The detector 30 utilizes a 1×1 mm active area, has a D*>2.5E+9 cmHz^{1/2}/W and a response time <3 ns. This system rules out physiological interferences and baseline shifts observed during clinical validation. The applicant used Partial Least Squares (PLS), a commonly used multivariate spectral processing technique, to develop the calibration and prediction model to accurately quantify glucose in blood. The applicant also used spectral preprocessing and digital filtering techniques to further enhance the glucose specific information by removing baseline offsets and high frequency noise.

The applicant derived the optimal path length 14 (FIG. 1) for the system. As the path length 14 of the transmission cell 33 increases, the absorption due to the materials within the cell 33 will also increase. The strongest absorber of light 36 within the mid-infrared region is water. Eventually, water absorption peaks completely mask all resolvable analyte peaks if the path length 14 is continuously increased. The ideal path length 14 is one which allows for resolvable differences between physiological concentrations of the analyte such as glucose.

In the vast majority of cases, infrared-based analytical methods are developed via calibration to accepted reference analyses. Calibration therefore derives a model which can recover quantitative analytical information from the infrared spectra. Although this step is a trivial one for very simple (one or two component) systems, more complex mixtures (matrix) require a more sophisticated approach.

The general procedure is the same regardless of the details of the process. The first stage is to accumulate both infrared spectra and assays for a set of appropriate clinical specimens. Ideally, this set of calibration samples should span the range of concentration expected both for the analyte of interest and for any interfering species (i.e. any absorber other than the target compound). Separate calibration models are then developed for each of the target analytes. Finally, each of the calibration models is validated by comparing infrared-predicted levels to the reference levels determined for an independent set of test specimens.

The three of the more common techniques are: multi-wavelength linear regression (MLR), principal component regression (PCR) and partial least squares (PLS). The MLR technique is an extension of Beer's law to include multiple wavelengths and has been described earlier (Equation 2). While simple and powerful, this technique is not guaranteed to find the optimal set of wavelengths in a spectral region, particularly for complex specimens where many wavelength terms may be required, such as for whole blood.

The feature common to both PCR and PLS approach is that each spectrum is reduced to a sum of pseudo-spectra, or “loading vectors”. Each spectrum is newly represented by a unique set of “scores”—the set of coefficients required to reconstruct the original spectrum from the set of loading vectors. Typically 5-15 loading vectors replace the thousands of intensity values in the original spectra. These scores then provide the basis for quantitation. The essential relationship in both the PCR and PLS models take the form of:

A=TB+EA Equation 4:

With m spectra in the calibration set, each having n absorbance values, A is the m×n matrix of the calibration spectra. The spectra are reconstructed as a product of B (h×n), the new basis set of loading vectors, and T (m×h), the scores. The key to the process is that each spectrum is reduced from a vector of length n (a row in A) to a new vector of length h (the corresponding row in T), where h is typically between 5 and 15. E_{A} corresponds to the spectral residuals. The column matrix of concentration c is also related to the loading vectors T, according to:

c=Tv+e_{c} Equation 5:

Here, v is the matrix of coefficients that relates the scores to the concentrations.

The selection of appropriate optical, fluidic and electronic components and their operating characteristics has been relevant in the successful development of this system. Monitor characterization had involved identifying the laser 21 and its operating conditions (such as pulse frequency, duty cycle, power and temperature of the thermoelectric cooler 31), evaluating the performance of the photoconductive detector in terms of detectivity and noise characteristics, determination of SNR (signal to noise ratio) for the dynamic range of glucose, determination of the appropriate optical path length 14 for maximum sensitivity, and assessment of wavelength requirements.

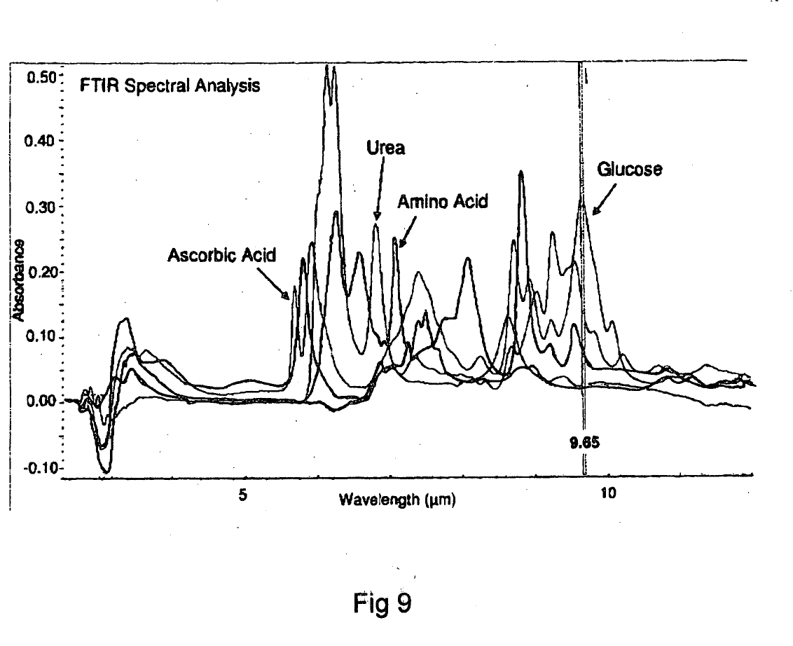

FIG. 9 shows the spectra of various common interferents found in whole blood. The graph shows that glucose has a sharp peak at 9.65 microns with minor interferences due some of the compounds. This spectral analysis helped in selecting appropriate wavelengths in the mid-infrared region for extracting the glucose specific information for accurate quantification.

The recent commercial availability of mid-infrared quantum cascade lasers (QCL) have changed the landscape of potential mid infrared based sensing applications. The QCLs can be operated at room temperature (without cryogenic cooling) conditions and have orders of magnitude better performance in terms of optical power and efficiency than traditional black body sources as in FTIR spectrometers.

A QCL is a unipolar semiconductor laser where light generation is based on intersubband transitions within the conduction band (or valence band). In contrast, conventional semiconductor lasers are bipolar devices where the light generation is based on the recombination of electrons from the conduction band and holes from the valence band across the band gap. Therefore, while the semiconductor material determines the laser wavelength, most common being AlGaAs semiconductors, the emission wavelength of a QCL is determined by the thickness of the alternating layers of different semiconductor materials. The QCLs can be mass-produced leading to inexpensive products.

QCLs have successfully been used for gas absorption measurements and photo-acoustic spectroscopy. In liquid phase, a room temperature QCL can operate with optical path lengths 14 of more than 100 μm, even in the case of aqueous matrices. Furthermore, using room temperature QCLs the signal-to-noise ratio was improved by a factor of 50 compared to state-of-the-art FTIR spectrometers.

High absorbency due to the presence of hemoglobin (100 times higher concentration than glucose), in addition to high water absorption in the mid-infrared region, turns out to be advantageous in laser based spectroscopic analysis because only micro-liters of blood are required to form a thin film of liquid in the sampling cell. Thus, by utilizing a transmission cell 33, referring to FIG. 1, which samples only a few micro liters of blood, a thin film of liquid 11 is created that is appropriate for sensing.

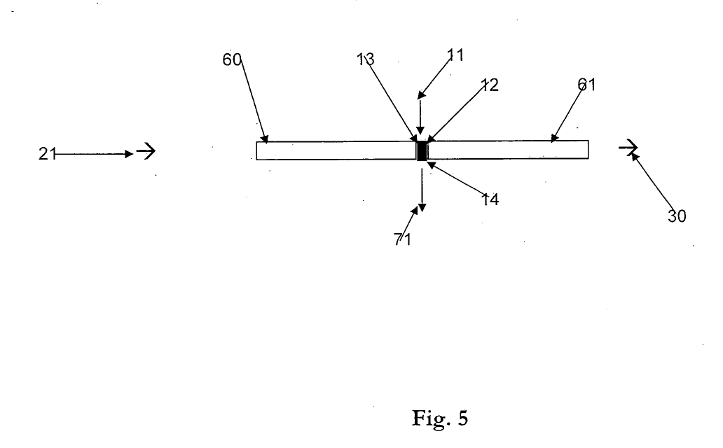

However, in order to keep a constant film 11 in the transmission cell 33, it has been found that a non-ionic surfactant (not shown) must be added to the whole blood sample 11 at a concentration range of about 0.1%-10% to reduce the surface tension and to lyse red blood cells and other cells that can cause noise in the reading due to optical instabilities. Examples of such surfactants include, Triton X-100 and Saponin. Referring now to FIG. 5, according to another embodiment of the present invention, a continuous whole blood glucose monitor, comprising: a sensor fluidic interface with a patient, a transmission cell 33, a single lumen catheter 40, a tube 84, a surfactant-saline supply 50, a mixer 41 and a pump 32: the sensor fluidic interface comprising: a laser 21, a detector 30, a fiber coupled transmission probe (High Tech Photonics, FL) 70 comprising fiber bundles (not shown); one end of the fiber bundles is connected to the detector 30 and laser 21, the other end of the fiber bundles is proximal to a mil or 10 to reflect the light from the laser 21 back to the detector 30; a transmission cell 33 is fixed between the mirror 10 and fiber-coupled transmission probe 70, a distance defining the path length 14 of the transmission cell. The fiber referred to throughout this application is suitable for transmitting mid-infrared light and is made up of Silver Halide material.

One end of the catheter 40 is inserted into a patient's peripheral vein, the other catheter 40 end is connected to the surfactant-saline supply 50, one end of the tube 84 is connected to the surfactant and saline supply 50, the other ends of the catheter 40 and the tube 84 are connected to the mixer 41 and pump 32, which carries fixed and metered amounts of the blood sample mixed with fixed and meter ed amounts of the surfactant-saline supply 50 through the transmission cell 33. Another advantage of a higher intensity light source is the ability to channel the light through a fiber optic system with high efficiency. Other advantages of QCLs in this invention include their small size, possibility for hybrid integration, narrow wavelength selectivity due to spectral line width and mechanical robustness. A tunable QCL can be used for simultaneous detection of multiple analytes, which have characteristic absorption in the mid-infrared spectral region.

While whole blood is preferred as the bodily fluid, as described, other fluids include plasma, serum (i.e. cell free blood) and blood ultrafiltrate (i.e. cell and large protein fi-ee). Blood serum is blood plasma from which clotting factors have been removed. There are various methods for continuous extraction of plasma and ultrafiltrate from whole blood. Neither of these methods would require sample homogenization by cell lysis as described for whole blood. One possible Plasma Extraction Method would be to employ a porous membrane to harvest roughly half of the serum-plasma from the patient blood sample in a flow by operation where the filter membrane comprises the walls of a flow channel continuously extracting serum-plasma, while the blood flows on its way to the waste container. Then the plasma sample is interrogated. Membrane geometry and the differential pressure across the membrane must be controlled to harvest sufficient plasma for measurement while leaving enough to avoid plugging of the membrane. Plugging of the membrane by blood cells must be avoided or accommodated by controlled back flush of plasma. Flow rate of plasma must be sufficient to minimize lag time between blood withdrawal and glucose measurement.

As to utilizing a suitable Ultrafiltration extraction method, Ultrafiltrate may be obtained from the subcutaneous space saline using ultrafiltration fibers available from Bioanalytical System, IN. However, because of the time associated with interstitial fluid and the travel time, this system is not suitable for use in hospitals. A better approach would be to obtain ultrafiltrate samples derived directly from vascular system. Hemofiltration is a well known method to obtain ultrafiltrate.

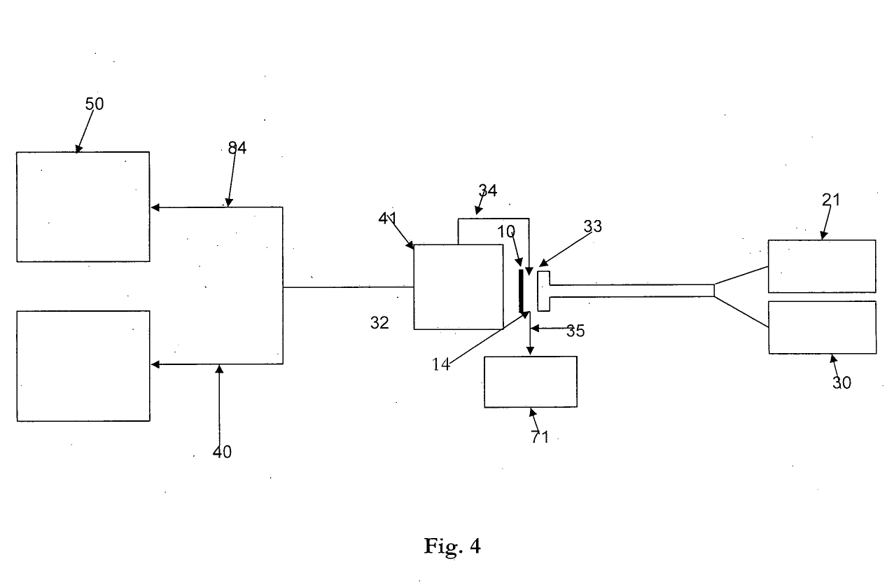

Referring now to FIG. 4, another embodiment of the present invention utilizes liquid-phase detection of glucose in body fluids. In this embodiment, the system comprises a quantum cascade laser 21, a detector 30, a first fiber coupled transmission probe 60 and a second fiber coupled transmission probe 61. Each transmission probe (60 and 61) comprising first and second ends, and containing fiber bundles (not shown) or wave guides extending from the first end of each transmission probe (60 and 61) to the second end of the transmission probe (60 and 61) respectively. Two transparent windows 13 and 12 spaced apart form a path length 14. One window 13 is connected to the first transmission probe 60 second end. The other window 12 is connected to the second transmission probe 61 first end. A flow cell 33 is positioned between the two windows 13 and 12. A tube manifold (not shown) for mixing a blood sample, an anti-coagulant, and a surfactant, is provided. The first end of first transmission probe 60 is proximal to the laser 21. The second end of the second transmission probe 61 is connected to the detector 30, A single lumen catheter 40, and tube 84 (as in FIG. 5), and an anti-coagulant surfactant supply.

One end of the catheter is inserted into a patient's peripheral vein. One end of the tube 84 is connected to the surfactant-saline supply 50. The other ends of the catheter and the surfactant-saline supply 50 are connected to the mixer 41 and pump 32. The pump 32 carries fixed and metered amounts of the blood sample mixed with fixed and metered amounts of the surfactant-saline supply 50 through the transmission cell 33. The light transmits from the laser 21 through the first transmission probe 60 through the flow cell 33, through the second transmission probe 61 to the detector 30.

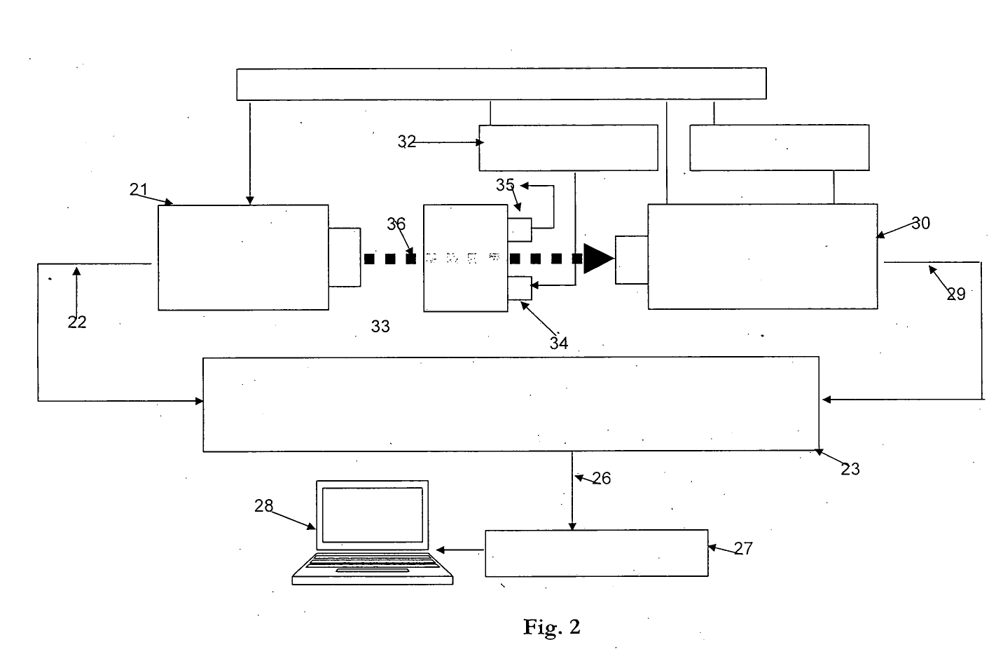

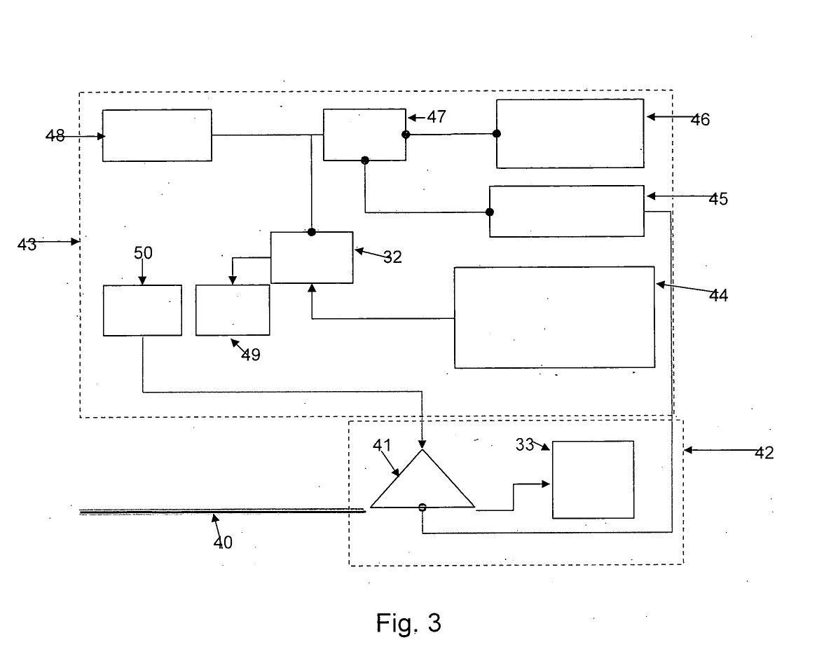

Referring again to FIG. 2 and 3, another preferred embodiment comprises a method of using the system. A method for monitoring glucose levels in whole blood and other biological fluids like plasma or ultrafiltrate in patients, comprises the step of connecting a catheter 40 to a peripheral vein of a patient and a fluid mixing valve 41; the step of connecting a tube 84 to a non-ionic surfactant and saline supply 50 and the mixing valve 41; the step of connecting the mixing valve 41 to the peristaltic pump 32; the step of connecting the peristaltic pump 32 to a transmission cell 33 having a path length 14 sized to resolve the physiological concentrations of the analyte within the sample; the step of integrating a processor 47 with the peristaltic pump 32 and mixing valve 41 to draw fixed and metered amounts of both the matrix sample and of the non-ionic surfactant and saline 50; the step of positioning the transmission cell (flow cell) 33 in the optical electromagnetic radiation path 36 of a mid-infrared quantum cascade laser 21 and a photo-detector 30 integrated with hardware and an algorithm configured to calculate the analyte concentration, and displaying the results on a computer 28; the step of calibrating the laser 21 and photo-detector 30.to specify the laser intensity 36 and the optimal set of wavelengths in a spectral region for the complex matrix where many wavelength terms may be required; the step of activating the fluidic system to first draw metered samples of the matrix and the saline and surfactant 50; then to mix the sample with the surfactant and saline 50; then to carry the mixture to the transmission cell 33; then after the optical measurement, rinse the transmission cell 33 with the surfactant and saline 50 to prevent clogging and non-homogeneity on the surfaces of the transmissive windows (13 and 12) during pumping the mixed sample through the transmission cell 33; the non-ionic surfactant helping to keep a constant film 11 in the transmission cell 33, by reducing surface tension, by lysing red blood cells and other cells that can cause optical instabilities in the measurement reading, by solubilizing proteins, and by homogenizing the matrix sample; and the step of activating the laser trigger to shoot many shots through the transmission cell 33 while the sample matrix passes.

Various proof-of-principle studies have been performed on the preferred embodiment of the quantum cascade laser based sensor system. The idea was to simulate real life conditions by monitoring changes in the glucose specific signal while continuously pumping randomly selected glucose-doped samples through the flow-cell. The sample matrix was made progressively complex from serum to whole blood. A clinically relevant dynamic range of 0-500 mg/dl was selected to monitor the real-time sensor response. The results clearly show that the sensor prototype can accurately resolve clinically relevant changes in glucose concentration with high sensitivity over the entire dynamic range.

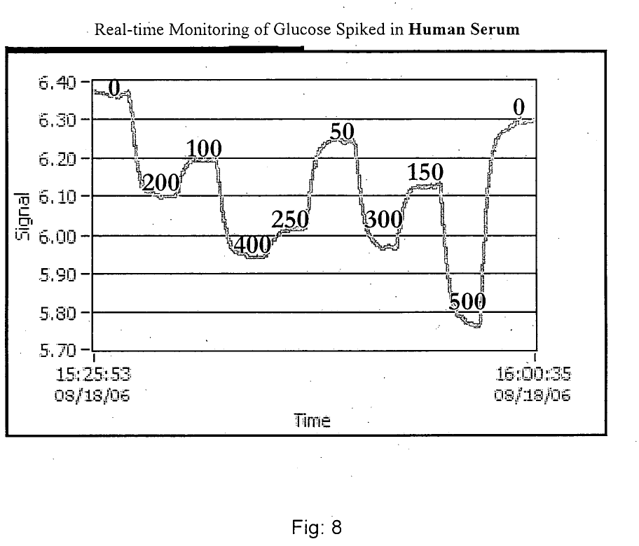

Referring to FIG. 8, the biological sample was human serum depleted of glucose (American Biological Technologies, TX). This allowed evaluating sensor performance in both hypo and hyperglycemic regions. The base glucose concentration was negligible and all higher concentrations were prepared by spiking with glucose stock solution. Real-time sensor response was observed while sequentially introducing serum samples having different glucose concentrations.

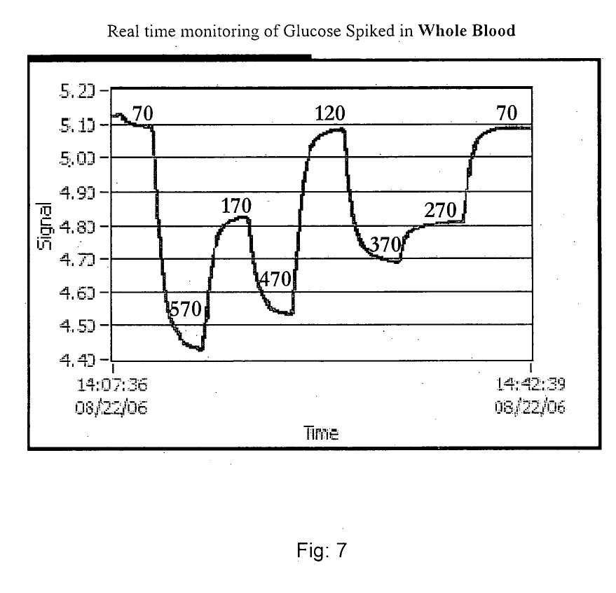

Referring to FIG. 7, whole blood was withdrawn into blood collection tubes from a healthy human subject. The tubes were pooled together and the blood cells were allowed to metabolize the existing glucose to achieve a low blood glucose concentration of 70 mg/dl. Higher glucose concentrations were prepared by adding D-glucose in 2 ml aliquots. 50 μl of 2% Triton X surfactant nixed in saline buffer was added to the 2 ml aliquots of whole blood to prevent clogging along the flow path and sample homogenization by lysis. Each sample was sequentially introduced using a peristaltic pump through the flow-cell. The transmitted signal changes were observed in real-time.

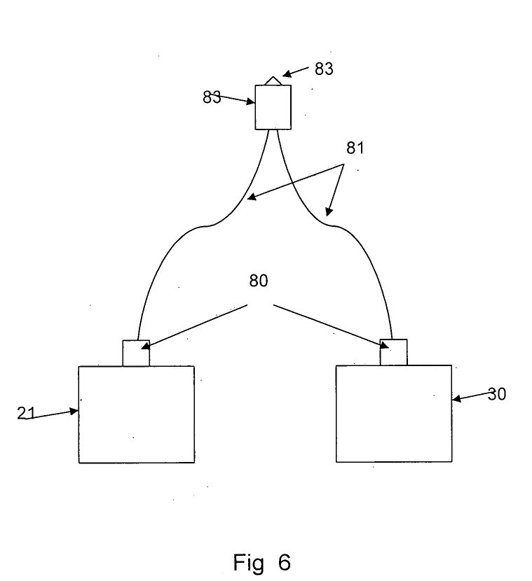

Referring now to FIG. 6, another preferred embodiment comprises a Mid-infrared quantum cascade laser 21 and a photo detector 30, each having collimating lenses 80; an ATR ZnSe crystal prism 82 having a tip 83 the size of a pinhead; and a silver halide Mid-infrared fiber 81 connecting the laser 21 and detector 30 with the ATR prism 82; which ATR prism 82 remotely interfaces with a patient's bodily fluid for glucose determination; the laser 21 Mid-infrared, electromagnetic signals bounce off the tip 83 through the fiber 81, reflecting back to the detector 30; the measurement sensitivity is limited by the tip 83 design. For example a hemispherical shaped prism tip increases the path length by increasing the number of optical bounces.

While the invention has been disclosed in preferred forms, it will be apparent to those skilled in the art that many modifications, additions, and deletions may be made therein without departing from the spirit and scope of the invention as set forth in the following claims.

What is claimed is:

1. A system for measuring an analyte in a complex matrix, comprising: a mid-infrared quantum cascade laser emitting at least one wavelength; a photo-detector; a means for exposing a sample of the complex matrix to the laser and photo detector; and a means for enhancing measurement sensitivity.

2. The system in claim 1, wherein the analyte is glucose.

3. The system in claim 2, wherein the matrix is selected from the group consisting of whole blood, plasma, serum, and ultrafiltrate.

4. The system in claim 1, wherein the system further comprises a thermo-electric cooler; the laser and the photo-detector operate at room temperature being cooled by the thermoelectric cooler.

5. The system in claim 4, wherein: the quantum cascade laser contains an external trigger that emits many mid-infrared electromagnetic radiation shots; and the means for enhancing measurement comprises electronics; including a time gate that coincides with the trigger; a gated integrator designed to recover fast, repetitive, analog signals; a boxcar averager with an active baseline subtraction module; the photo detector converts the many radiation signals to analog signals and feeds the analog signals to the gated integrator that amplifies and integrates the analog signals present when the gate is open and ignores noise and interference during other times; the boxcar averager averages the output of the gated integrator, and the active baseline subtraction module cancels baseline drift.

6. The system in claim 5, wherein the means for exposing the sample to the photo detector, comprises a fluidic system comprising, a transmission cell, a peristaltic pump, and a catheter, and a saline supply for rinsing the transmission cell.

7. The system in claim 6, wherein the transmission cell has a pair of mid-infrared transmissive windows spaced apart in the path of the mid-infrared electromagnetic radiation shots and the photo detector; the peristaltic pump delivers the sample from the catheter to the transmission cell; the spacing between the windows defines the optimal path length that enhances measurement sensitivity.

8. The system in claim 7, wherein the transmissive windows are preferably Zinc Selenide, but not excluding Zinc Sulfide, Silicon, and Polypropylene.

9. The system in claim 8, wherein one end of the catheter is inserted in a peripheral vein of a patient, whereas the other catheter end is connected to the peristaltic pump.

10. The system in claim 9, wherein the analyte is glucose, and the catheter dimensions are also sized to minimize blood consumption and lag time between whole blood withdrawal and glucose measurements.

11. The system in claim 10, wherein a method for preventing clogging and non-homogeneity of the matrix sample, and for preventing air bubbles in the transmission cell, comprises the step of using known methods in the industry.

12. The system in claim 10, wherein the fluidic system contains a non-ionic surfactant and saline supply, a catheter, a tube, and a mixing valve; the catheter is inserted into a patient, for extracting a fixed and metered sample of the matrix, and is connected to the mixing valve; the tube connects a fixed and metered amount of the surfactant and saline supply to the mixing valve; both the sample and the matrix and the saline and surfactant are metered so that a dilution factor is controlled; the peristaltic pump is connected to the mixing valve and the transmission cell;
the fluidic system draws metered samples of the matrix and the saline and surfactant; then the fluidic system mixes the sample with the surfactant and saline; then the peristaltic pump carries the mixture to the transmission cell; then after the optical measurement, the transmission cell is rinsed with the surfactant and saline to prevent clogging and non-homogeneity on the surfaces of the transmissive windows during pumping the mixed sample through the transmission cell; the non-ionic surfactant keeps a constant film in the transmission cell, by reducing surface tension, by lysing red blood cells and other cells that can cause optical instabilities in the measurement reading, by solubilizing proteins, and by homogenizing the matrix sample.

13. the system in claim 12, wherein the surfactant has a concentration range of about 0.1%-10%.

14. The system in claim 13, wherein the non-ionic surfactant is selected from the group consisting of Triton X-100 and Saponin.

15. The system in claim 14, wherein a heparin coating coats the inside surfaces of the fluidic system in contact with the sample matrix; the heparin coating prevents clot formation along the flow path.

16. The system in claim 15, wherein the heparin coating may be of a covalent linkage type selected from the group consisting of the CBAS coating offered by Carmeda, and other complexes such as the TDMAC-Heparin Complex, and Heparin Benzalkonium Chloride Complex.

17. The system in claim 16, wherein in the catheter is a single lumen peripheral intravenous blood access catheter.

18. The system of claim 17, including signal processing hardware which converts the mid-infrared laser beam into electronic signals, and enhances the signal to noise ratio.

19. The system of claim 18, including a data acquisition card which converts the electronic signals into computer readable data; and a glucose prediction application which reads the data, calculates the glucose concentration in the whole blood sample and displays the results on a computer.

20. The system of claim 19, wherein the infrared transmissive windows are a pair of circular windows made of a material selected from the group consisting of Zinc Selenide or Polypropylene with small optical apertures; the spaced apart windows from a path length from microfluidic interface of a matrix sample and the transmission cell; the path length is sized to allow the mid-infrared electromagnetic radiation signal to pass through the matrix sample at an optimal intensity enhancing measurement sensitivity.

21. The system in claim 20, wherein the laser is a tunable, multi-wavelength, mid-infrared quantum cascade laser.

22. The monitor of claim 23, wherein the tunable quantum cascade laser is centered at 9.4 μm having a ±5 percent tunability around the center wavelength.

23. A method for monitoring an analyte in a complex matrix, comprising:
the step of connecting a catheter to a peripheral vein of a patient and a fluid mixing valve;
the step of connecting a tube to a non-ionic surfactant and saline supply and the mixing valve;
the step of connecting the mixing valve to the peristaltic pump;
the step of connecting the peristaltic pump to a transmission cell having a path length sized to resolve the physiological concentrations of the analyte within the sample;
the step of integrating a processor with the peristaltic pump and mixing valve to draw fixed and metered amounts of both the matrix sample and of the non-ionic surfactant and saline;
the step of positioning the transmission cell in the optical electromagnetic radiation path of a mid-infrared quantum cascade laser and a photo-detector integrated with hardware and an algorithm configured to calculate the analyte concentration, and displaying the results on a computer;
the step of calibrating the laser and photo-detector to specify the laser intensity and the optimal set of wavelengths in a spectral region for the complex matrix where many wavelength terms may be required;
the step of activating the fluidic system to first draw metered samples of the matrix and the saline and surfactant; then to mix the sample with the surfactant and saline; then to carry the mixture to the transmission cell; then after the optical measurement, rinse the transmission cell with the surfactant and saline to prevent clogging and non-homogeneity on the surfaces of the infrared transmissive windows during pumping the mixed sample through the transmission cell; the non-ionic surfactant helping to keep a constant film in the transmission cell, by reducing surface tension, by lysing red blood cells and other cells that can cause optical instabilities in the measurement reading, by solubilizing proteins, and by homogenizing the matrix sample; and
the step of activating the laser trigger to shoot many shots through the transmission cell while the sample matrix passes.

24. The method in claim 23, wherein the analyte is glucose and the complex matrix is selected from the group consisting of whole blood, plasma, and ultrafiltrate.

25. A method of blood sampling, comprising:
the step of inserting a single lumen catheter into a patient's peripheral vein;
the step of withdrawing blood continuously or on demand by a peristaltic pump operating at a low speed to prevent any vein collapse and coating the fluidic path with heparin to prevent blood clotting.

26. A plasma extraction method, comprising:
the step of employing a porous membrane to harvest roughly half of the serum-plasma from the patient blood sample in a flow by operation where the filter membrane comprises the walls of a flow channel continuously extracting serum/plasma while the blood flows on its way to the waste container;
the step of interrogating the plasma sample;
the step of controlling the membrane geometry and the differential pressure across the membrane to harvest sufficient plasma for measurement while leaving enough to avoid plugging of the membrane;
the step of back flushing applying a controlled back flush of the plasma to avoid plugging of the membrane by blood cells; and
the step of regulating the flow rate of plasma sufficient to minimize lag time between blood withdrawal and glucose measurement.

27. An ultrafiltration extraction method, comprising: the steps selected from the group consisting of the step of using ultrafiltration fibers to obtain ultrafiltration from the subcutaneous space of a patient or, the step of obtaining ultrafiltrate samples derived directly from vascular system by using hemofiltration.

28. A modular continuous whole blood glucose monitor, comprising: a mid-infrared optical subsystem, including a tunable multi-wavelength mid-infrared quantum cascade laser, photo detector, electronics, and software; and a microfluidic cartridge, including a transmission cell, a fluid selector valve, and blood access catheters.

29. The monitor in claim 27, wherein the monitor is portable, the microfluidic cartridge is separate from the optical subsystem, the microfluidic cartridge is disposable and insertable into the optical subsystem.

30. The monitor in claim 28, wherein tile microfluidic cartridge displays glucose values both graphically and numerically.

31. The monitor in claim 29, wherein the monitor is rechargeable and battery operated.

32. The monitor in claim 30, including an alarm that activates whenever the glucose levels fall out of the safe range.

33. The monitor in claim 31, including a wireless transmitter capable of sending data, in a open or closed loop, to ancillary systems to help maintain tight glycemic control.

34. The monitor in claim 32, wherein life cycle of the microfluidic cartridge and the catheters set is at least 3 days, the typical time period a patient remains in ICU.

35. A method for determining optimal wavelengths of the multi-wavelength laser in claim 33, comprising:
the step of reducing each spectrum to a sum of pseudo spectra loading vectors;
the step of representing each spectrum by a unique set of scores, the set of coefficients required to reconstruct the original spectrum from the set of loading vectors using equation: A=TB+E_{A}; wherein A is the m×n matrix of the calibration spectra; wherein m spectra in the calibration set, each having n absorbance values; wherein the spectra are reconstructed as a product of B (h×n), the new basis set of loading vectors, and T (m×h), the scores; and,
the step of using the equation: c=Tv+e_{c} to relate the unique set of scores to c the column matrix of concentrations.

36. The monitor in claim 34, wherein the microfluidic cartridge is a fluidic system further comprising, mixing valve, a saline and non-ionic surfactant supply, and a heparin coating; the transmission-cell including a pair of circular windows made of material selected from the group consisting of Zinc Selenide or Polypropylene with small optical apertures, spaced apart foiling a pathlength for microfluidic interface of a whole blood sample and the transmission cell; the path length being sized to allow the mid-infrared laser electromagnetic signal to pass through the whole blood sample at an optimal intensity;
the catheter is sized to minimize blood consumption and lag time between whole blood withdrawal and glucose measurements; all the inside surfaces of the fluidic system are coated with the heparin;
the catheter is inserted into a patient, for extracting a fixed and metered sample of the matrix, and is connected to the mixing valve; the tube connects a fixed and metered amount of the surfactant and saline supply and mixing valve; both the sample and the matrix and the saline and surfactant are metered so that a dilution factor is controlled; the peristaltic pump is connected to the mixing valve and the transmission cell.

37. A continuous whole blood glucose monitor, comprising: a sensor fluidic interface with a patient, a transmission cell, a single lumen catheter, a tube, a surfactant-saline supply, a mixer and a pump: the sensor fluidic interface comprising: a laser, a detector, a fiber coupled transmission probe comprising fiber bundles; one end of the fiber bundles is connected to the detector and laser, the other end of the fiber bundles is proximal to a mirror to reflect the light from the laser back to the detector; a transmission cell is fixed between the mirror and fiber-coupled transmission probe, a distance defining the path length of the transmission cell;
one end of the catheter is inserted into a patient's peripheral vein, the one end of the tube is connected to the surfactant-saline supply, the other ends of the tube and catheter are connected to the mixer and pump; the pump carries fixed and metered amounts of the blood sample mixed with fixed and metered amounts of the surfactant-saline supply through the transmission cell.

38. A continuous whole blood glucose monitor, comprising: a fiber coupled transmission fluidic inter face with a patient; the interface comprising a mid-infrared quantum cascade laser, a detector, a first fiber coupled transmission probe and a second fiber coupled transmission probe, each transmission probe comprising first and second ends, and containing fiber bundles or wave guides extending from the first end of each transmission probe to the second end of the transmission probe respectively; two transparent windows spaced apart forming a path length; one window connected to the first transmission probe second end, the other window connected to the second transmission probe first end; a transmission cell in between the two windows; a tube manifold for mixing a blood sample, an anti-coagulant, and a surfactant; the first end of first transmission probe is proximal to the laser, the second end of the second transmission probe is connected to the detector; a double lumen catheter, and a anti-coagulant surfactant supply;
one end of the catheter is inserted into a patient's peripheral vein, the one end of the tube is connected to the surfactant-saline supply, the other ends of the tube and catheter are connected to the mixer and pump; the pump carries fixed and metered amounts of the blood sample mixed with fixed and metered amounts of the surfactant-saline supply through the transmission cell, the light transmits from the laser through the first transmission probe through the flow cell, through the second transmission probe to the detector.

39. The monitor of claim 34, including a Mercury Cadmium Telluride detector package, comprising; a response time of less than 9 nanosec, a detectivity of 7E9 cmHz^{1/2}/W, and a field of view of 38°; a detector chip and a preamplifier integrated together; the Mercury Cadmium Telluride detector package provides shielding of the thermo-electric controller module;
the signal processing hardware comprising, a gated integrator module with a gate having a gate width; a boxcar averager; and a unique active baseline subtraction module; the gated integrator and boxcar averager being designed to recover fast, repetitive, analog signals; the gate width being sized and perfectly aligned with the external trigger on the quantum cascade laser driver, such that when the gate opens, the mid-infrared laser signal passes through the transmission cell and the gate, and is sensed by the Mercury Cadmium Telluride detector package, which feeds the mid-infrared laser signal into the gated integrator, which amplifies and integrates the mid-infrared laser signal while ignoring noise and interference that are present when the gate is closed; the gated integrator then feeds the amplified and integrated mid-infrared signal into the Boxcar Averager, which averages the amplified and integrated mid-infrared signal improving the signal-to-noise ratio by a factor of the square root of the number of shots sampled; and the active baseline subtraction module cancels baseline drift.

40. A bodily fluid monitor, comprising: a Mid-infrared quantum cascade laser and a photo detector, each having collimating lenses; an ATR ZnSe crystal prism having a tip the size of a pinhead; and a silver halide Mid-infrared fiber connecting the laser and detector with the ATR prism; which ATR prism remotely interfaces with a patient's bodily fluid for glucose determination; the laser Mid-infrared, electromagnetic signals bounce off the tip through the fiber, reflecting back to the detector; the measurement sensitivity is determined by the tip design.

41. The monitor in claim 40, wherein the ATR prism tip has a hemispherical geometry, which design increases the path length by increasing the number of optical bounces.

42. The monitor in claim 41, wherein the detector is cryogenically cooled made of MCT, whose cooling increases detectivity.

43. A continuous whole blood glucose monitor, comprising: a monochromatic, pulsed, multimode quantum cascade laser operating at around room temperature; the quantum cascade laser comprising, a driver with an external trigger, the driver capable of generating a mid-infrared laser signal having a wavelength of 9.65 μm, a pulse frequency of 10 kHz, a pulse width of 100 ns, a peak power of 1000 mW; a room temperature MCT detector package; a thermo-electric controller module for maintaining the monitor around room temperature; a fluidic system comprising a peristaltic pump, a demountable transmission based flow-cell, and a single lumen peripheral intravenous blood access catheter for transmitting a whole blood sample from a patient's peripheral vein to the flow-cell, a tube; signal processing hardware which converts the mid-infrared laser signals into analog signals; a data acquisition card which converts the analog signals into digital signals; and a glucose prediction algorithm which reads the data, calculates the glucose concentration in the whole blood sample and displays the results on a computer;
the quantum cascade laser and driver being in optical communication with the fluidic system and infrared integrated detector package;
the fluidic system further comprising, a fluid selector valve, a saline and surfactant supply, and a CBAS heparin coating having thickness of 0.2 μm and being non leaching, sterilizable, and hydrophilic; the flow-cell including a pair of circular ZnSe windows spaced apart approximately 100 microns, forming a path length for microfluidic interface of a whole blood sample and the flow cell; the path length being sized to allow the mid-infrared laser signal to pass through the whole blood sample at an optimal intensity; the flow cell having a contained volume of less than 10 μl;
the catheter comprising, a sterilized capillary tubing with an outside diameter of 360 μm, an inside diameter of 150 μm, an inside surface, a first catheter end and a second catheter end, and a 22 gauge sheath; the catheter dimensions minimize blood consumption and the lag time between whole blood withdrawal and glucose measurements; all the inside surfaces of the fluidic system are coated with the heparin coating to prevent platelet adhesion and thrombus formation on the inside surfaces, while not diluting the whole blood sample, while reducing risk of trauma to the vascular system of the patient, and while avoiding heparin induced disorders to the patient;
the catheter is inserted into a patient, for extracting a fixed and metered sample of the whole blood, and is connected to the fluid selector valve; the tube connects a fixed and metered amount of the surfactant and saline supply and mixing valve; both the sample and the whole blood sample and the saline and surfactant are metered so that a dilution factor is controlled; the peristaltic pump is connected to the fluid selector valve and the flow cell; the fluid selector valve alternates between the saline and surfactant supply and the whole blood sample, every 5 minutes or at other user defined measurement intervals;
the MCT detector package, comprising; a response time of less than 9 nsec, a detectivity of 7E9 cmHz^{1/2}/W, and a field of view of 38°; a detector chip and a preamplifier integrated together; the MCT detector package provides shielding of the thermo-electric controller module;
the signal processing hardware comprising, a gated integrator module with a gate having a gate width; a boxcar averager; and a unique Active Baseline Subtraction module; the gated integrator and boxcar averager being designed to recover fast, repetitive, analog signals; the gate width being sized and perfectly aligned with the external trigger on the quantum cascade laser driver, the mid-infrared laser beam passes through the flow cell, and is sensed by the MCT detector package, which package feeds the transmitted signal into the gated integrator, which gated integrator amplifies and integrates the mid-infrared laser signal while ignoring noise and interference that are present when the gate is closed; the gated integrator then feeds the amplified and integrated mid-infrared signal into the Boxcar Averager, which averager averages the amplified and integrated mid-infrared signal improving the signal-to-noise ratio by a factor of the square root of the number of shots; and the Active Baseline Subtraction module cancels baseline drift.
